(19) United States
(12) Patent Application Publication
(10) Pub. No.: US 2024/0325285 A1
(43) Pub. Date: Oct. 3, 2024

(54) ANTIBACTERIAL COMPOSITION

(71) Applicant: Kolmar Korea Co., Ltd., Sejong (KR)

(72) Inventors: Hye Young YOO, Seoul (KR); Sung Ha PARK, Seoul (KR); Dong Min SHIN, Seoul (KR); Yong Hyun KIM, Seoul (KR); Eun Hye LEE, Seoul (KR); Eun Sung KO, Seoul (KR)

(21) Appl. No.: 18/572,618

(22) PCT Filed: Jul. 25, 2023
(86) PCT No.: PCT/KR2023/010728

§ 371 Date: Dec. 20, 2023

(30) Foreign Application Priority Data
Mar. 24, 2023 (KR) 10-2023-0038604

(51) Int. Cl. A61K 8/92; A61K 36/185; A61K 36/53; A61K 36/899; A61Q 17/00

(52) U.S. Cl. CPC A61K 8/922; A61K 36/185; A61K 36/53; A61K 36/899; A61Q 17/005

(57) ABSTRACT

Provided is an antibacterial composition including lemongrass oil, lavender oil, and ylang ylang oil.

### BACKGROUND

### Field of the Invention

The present invention relates to an antibacterial composition.

### Discussion of Related Art

Recently, interest has been focused on the development of antibacterial substances derived from natural substances. These antibacterial substances are known to exhibit high antibacterial effects by combining various components derived from natural substances, and have low toxicity to humans or animals.

In addition, antibacterial substances play an important role in the medical or cosmetic fields and are specifically used in various cosmetics to solve problems related to skin diseases. For example, various antibacterial cosmetics are being developed to improve skin problems such as hair loss, dandruff, and acne, and these cosmetics are capable of minimizing the growth of harmful bacteria present on the skin by using antibacterial substances.

### SUMMARY OF THE INVENTION

The present invention is designed to solve the problems of the related art, and it is an object of the present invention to provide an antibacterial composition.

However, the technical objectives of the present invention are not limited to that described above, and other unmentioned technical objectives will be clearly understood by those skilled in the art from the following description.

According to one aspect of the present invention, there is provided an antibacterial composition including lemongrass oil, lavender oil, and ylang ylang oil.

In addition, a weight ratio of the lavender oil and the lemongrass oil may be 1:1.3 to 2.7.

Additionally, a weight ratio of the ylang ylang oil and the lemongrass oil may be 1:4 to 8.

In addition, a weight ratio of the ylang ylang oil and the lavender oil may be 1:1.5 to 4.5.

Additionally, a weight ratio of the ylang ylang oil, the lavender oil, and the lemongrass oil may be 1:1.5 to 4.5:4 to 8.

In addition, the composition may have antibacterial activity against at least one of strains of the genus Staphylococcus, the genus Streptococcus, the genus Malassezia, the genus Propionibacterium, and the genus Candida.

According to another aspect of the present invention, there is provided a cosmetic composition including the antibacterial composition.

According to still another aspect of the present invention, there is provided a composition for external use on the skin, including the antibacterial composition.

According to yet another aspect of the present invention, there is provided a pharmaceutical composition for preventing or treating hair loss, including the antibacterial composition.

### BRIEF DESCRIPTION OF THE DRAWINGS

In order to fully understand the drawing mentioned in the detailed description of the present invention, a brief description of the drawing is provided.

FIG. 1 is an image showing results of the minimum inhibitory concentration (MIC) measurement experiment of the present invention.

### DETAILED DESCRIPTION OF EXEMPLARY EMBODIMENTS

Unless otherwise defined, all technical and scientific terms used herein have the same meaning as commonly understood by those skilled in the art. In general, the nomenclature used herein is well known and commonly used in the art. Further, in the description of embodiments of the present invention, a detailed description of known functions and configurations incorporated herein will be omitted to avoid making the subject matter of the present invention unclear. Further, although embodiments of the present invention are described below, the present invention may be embodied in many alternate forms by those skilled in the art without departing from the spirit of the present invention and should not be construed as limited to only the embodiments set forth herein.

In the present specification, a certain part including a certain element signifies that the certain part may further include, instead of excluding, another element unless specifically indicated otherwise. In the present specification, the term “and/or” includes any and all combinations of a plurality of associated listed items.

In the present invention, “oil” refers to natural vegetable oil extracted from a plant and is used in the same sense as “essence,” “distilled extract,” or “essential oil.” The plant used for oil preparation may be a raw material that has not been subjected to a pulverizing or grinding process or a cut material that has been subjected to a pulverizing or grinding process, and include all parts such as roots, leaves, stems, flowers, fruits, and the like.

In the present invention, oil may be extracted through steam distillation, which is a common method of extracting the aromatic active ingredient of plants with vapor and condensing the resulting extract through cooling with cooling water. Also, oil includes a diluted or concentrated liquid of an extract obtained by extracting a plant through steam distillation, a dried product obtained by drying the extract, a crude product or purified product of the extract or a mixture thereof, the extract itself, and extracts of all formulations that can be formed using the extract. However, the present invention is not limited thereto.

In the present invention, oil may be in the form of a liquid, a solid made by drying a liquid-type extract, or a powder made by pulverizing a solid-type extract. In this case, as the drying method, various methods commonly used in the art, such as spray drying, freeze drying, vacuum drying, hot air drying, and the like, may be used. However, the present invention is not limited thereto.

According to one aspect of the present invention, there is provided an antibacterial composition including lemongrass oil, lavender oil, and ylang ylang oil. The antibacterial composition according to an embodiment of the present invention exhibits the synergistic effect of the oils by using a mixture of three types of oils, and thus antibacterial activity can be improved compared to a case in which one or two types of oil is/are included.

Lemongrass oil is an oil extracted from camel grass (Cymbopogon Schoenanthus), is also called camel grass oil (Cymbopogon Schoenanthus oil), and is known to have antifungal activity, disinfection, deodorization, or sterilization effects.

In an embodiment, the antibacterial composition may include the lemongrass oil in an amount of 30 to 85 wt %, specifically 43 to 75 wt %, more specifically 45 to 70 wt %, and even more specifically 50 to 70 wt % based on the total weight of the antibacterial composition.

Lavender oil is an oil extracted from lavender (Lavandula angustifolia) and is known to have antibacterial, anti-inflammatory, and antioxidant activities and have excellent skin protection from external harmful substances, soothing, or circulation effects.

In an embodiment, the antibacterial composition may include the lavender oil in an amount of 10 to 50 wt %, specifically 15 to 45 wt %, and more specifically 20 to 40 wt % based on the total weight of the antibacterial composition.

Ylang ylang oil is an oil extracted from the ylang ylang (Cananga odorata) tree and is known to alleviate skin and scalp symptoms caused by excessive sebum secretion by helping to balance sebum secretion, and have a hair strengthening effect.

In an embodiment, the antibacterial composition may include the ylang ylang oil in an amount of 1 to 30 wt %, specifically 3 to 25 wt %, and more specifically 5 to 20 wt % based on the total weight of the antibacterial composition.

For example, when the lemongrass oil, lavender oil, and/or ylang ylang oil is/are included in the above content range(s), excellent antibacterial effects, fragrance, feeling of use, and the like can be implemented. However, the present invention is not limited thereto.

In an embodiment, a weight ratio of the lavender oil and lemongrass oil may be 1:1.3 to 2.7. Additionally/alternatively, a weight ratio of the ylang ylang oil and lemongrass oil may be 1:4 to 8. Additionally/alternatively, a weight ratio of the ylang ylang oil and lavender oil may be 1:1.5 to 4.5. When the weight ratios of the lemongrass oil, lavender oil and/or ylang ylang oil fall within the above ranges, antibacterial activity can be enhanced. However, the present invention is not limited thereto.

In an embodiment, a weight ratio of the ylang ylang oil, lavender oil, and lemongrass oil may be 1:1.5 to 4.5:4 to 8, specifically 1:2 to 4:4 to 8, and more specifically 1:2.5 to 3.5:4 to 8. When the weight ratio of the lemongrass oil, lavender oil, and ylang ylang oil falls within the above range, antibacterial activity can be maximized to minimize hair loss, and excellent hair growth can be provided. However, the present invention is not limited thereto.

In an embodiment, the antibacterial composition may have antibacterial activity against harmful bacteria that cause a symptom such as hair loss, acne, or dandruff. For example, the antibacterial composition may have antibacterial activity against at least one of strains of the genus Staphylococcus, the genus Streptococcus, the genus Malassezia, the genus Propionibacterium, and the genus Candida, and specifically, antibacterial activity against a strain of the genus Staphylococcus.

In an embodiment, the antibacterial composition may have antibacterial activity against at least one of Staphylococcus aureus, Staphylococcus epidermidis, Streptococcus pyogenes, Malassezia furfur, Propionibacterium acnes, and Candida albicans, and specifically, antibacterial activity against Staphylococcus aureus.

According to another aspect of the present invention, there is provided a cosmetic composition. The cosmetic composition may include the above-described antibacterial composition.

In an embodiment, since the antibacterial composition has antibacterial activity against harmful bacteria that cause a symptom such as hair loss, acne, or dandruff, the cosmetic composition according to the present invention can alleviate a symptom such as hair loss, acne, or dandruff and promote hair growth.

In an embodiment, the cosmetic composition may further include an additive commonly used in the art within a range that does not impair the effect of the present invention. For example, the cosmetic composition may further include a thickener, a pH adjuster, an isotonic agent, a surfactant, a stabilizer, a preservative, a disinfectant, a moisturizer, a blocking agent, an antioxidant, an organic pigment, an inorganic pigment, a fragrance, a vitamin, and the like. The mixing amount of the above components may be easily selected by those skilled in the art within a range that does not impair the purpose and effect of the present invention.

In an embodiment, the cosmetic composition may be provided as any formulation commonly prepared in the art. For example, the cosmetic composition may be formulated in any form such as an oil dispersion, a water dispersion, an oil-in-water (O/W), water-in-oil (W/O), water-in-oil-in-water (W/O/W), or oil-in-water-in-oil (O/W/O) emulsion, a solubilized formulation, and the like. However, the present invention is not limited thereto, and the cosmetic composition may be provided in any form such as a solution, a suspension, an emulsion, a solid, a gel, an oil, a powder, a paste, and a foam aerosol.

In an embodiment, the cosmetic composition may be manufactured into various products. For example, the cosmetic composition may be manufactured into hair washing products such as a shampoo, a rinse, a hair conditioner, a hair gel, and the like or scalp and/or hair care products such as a scalp or hair toner, a scalp or hair essence, a scalp or hair serum, a scalp or hair tonic, and the like. In addition, the cosmetic composition may be manufactured into a soap, a cleaner, a cleansing cream, a cleansing water, and the like or manufactured into color cosmetics such as a lipstick, a lip balm, a mascara, a blusher, a shading, a highlighter, a makeup base, a foundation, a compact, a concealer, a skin cover, and the like. Additionally, the cosmetic composition may be manufactured into functional cosmetics such as a nourishing cream, an essence, and an ampoule or basic cosmetics such as a toner, a lotion, and the like. In addition, the cosmetic composition may be manufactured into products that are attached to the skin, such as a pack, a hydrogel slim patch, and the like, or products that are sprayed on the skin, such as a mist, an aerosol, and the like.

According to still another aspect of the present invention, there is provided a composition for external use on the skin. The composition for external use on the skin may include the above-described antibacterial composition.

The composition for external use on the skin refers to a preparation that externally acts on the skin among external preparations and may include products such as a drug, a quasi-drug, and the like, but the present invention is not limited thereto. For example, the composition for external use on the skin may be provided in any form such as a cream, a gel, a skin emulsifier, a patch, a spray, and the like.

In an embodiment, in the composition for external use on the skin, components commonly used in external skin preparations of cosmetics or drugs, for example, an aqueous component, an oil component, a powder component, an alcohol, a moisturizer, a thickener, a UV protective agent, a whitening agent, a preservative, an antioxidant, a surfactant, a fragrance, a coloring agent, a lipid material, a solubilizing agent, a thickener, a gelating agent, an emollient, a foaming agent, a fragrance, a sequestering agent, a chelating agent, a vitamin, various skin nutrients, and a combination thereof may be appropriately mixed as necessary. The mixing amount of the components may be easily selected by those skilled in the art within a range that does not impair the purpose and effect of the present invention.

According to yet another aspect of the present invention, there is provided a pharmaceutical composition for preventing or treating hair loss. The pharmaceutical composition may include the above-described antibacterial composition. “Prevention of hair loss” refers to partial or complete prevention, suppression, inhibition, and reduction of hair loss.

“Hair loss” refers to defective hair growth and partial or entire hair loss, and may include, for example, androgenetic alopecia, toxic alopecia, alopecia areata, telogen effluvium, alopecia caused by endocrine abnormalities, metabolic disorders, and nutritional disorders, chemotherapy-induced alopecia, mechanical alopecia, alopecia caused by skin diseases, cicatricial alopecia, congenital alopecia, and trichotillomania. For example, alopecia may have many etiologies including genetic factors, aging, local and systemic diseases, fever symptoms, mental stress, hormonal problems, and side effects of drugs.

In an embodiment, the pharmaceutical composition may promote hair growth. “Hair growth” may include maintaining, inducing, stimulating, promoting and regenerating hair development, growth of missing hair, extending the growth phase in the hair cycle, converting vellus hair into terminal hair, and the like. Also, “promotion of hair growth” may refer to promoting hair growth, extending the growth phase in the hair cycle, or converting vellus hair, which is a thin, smooth, short, and depigmented hair whose hair follicle is located on the surface of the dermis, into terminal hair, which is a thick, long, and pigmented hair whose hair follicle is deeply implanted in the dermis.

In an embodiment, the pharmaceutical composition may further include a pharmaceutically acceptable salt within a range that does not impair the effect of the present invention. Here, the “pharmaceutically acceptable salt” may be a salt that does not cause serious irritation to the organism to which the compound is administered and does not impair the biological activity and physical properties of the compound.

For example, the pharmaceutically acceptable salt may be formed of an inorganic acid and an organic acid, for example, hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetyl cysteine, or the like.

In addition, for example, the pharmaceutically acceptable salt may be prepared by adding an inorganic base or organic base to a free acid. A salt derived from an inorganic base may include, but is not limited to, sodium, potassium, lithium, ammonium, calcium, magnesium salts, and the like. A salt derived from an organic base may include, but is not limited to, salts of primary, secondary, and tertiary amines, substituted amines including naturally occurring substituted amines, cyclic amines, and basic ion-exchange resins, for example, isopropylamine, trimethylamine, diethylamine, triethylamine, tripropylamine, ethanolamine, lysine, arginine, N-ethylpiperidine, piperidine, polyimine resin, and the like.

In an embodiment, the pharmaceutical composition may further include a pharmaceutically acceptable carrier or additive within a range that does not impair the effect of the present invention. Here, the “pharmaceutically acceptable carrier” may be a compound that is commonly used in preparation, does not inhibit the activity of an active ingredient, does not have toxicity beyond what is acceptable for the subject of application or prescription, and facilitates the addition of the compound into cells or tissues.

In an embodiment, the pharmaceutical composition may be prepared in the form of a solid or liquid. For example, when prepared in the form of a solid, the pharmaceutical composition may further include a carrier, a flavoring agent, a binder, a preservative, a disintegrant, a lubricant, a filler, and the like. Also, when prepared in the form of a liquid, the pharmaceutical composition may further include water, a solution such as a propylene glycol solution, a suspension, an emulsion, and the like, but the present invention is not limited thereto. A colorant, a flavoring agent, a stabilizer, a thickener, and the like may be further included.

In an embodiment, the solid formulation may include a powder, a granule, a tablet, a capsule, a suppository, and the like. For example, the powder may be prepared by simply mixing a Undaria Pinnatifida Sporophyll extract, which is an active ingredient of the present invention, with a carrier such as lactose, starch, microcrystalline cellulose, or the like. The granule may be prepared by mixing a Undaria Pinnatifida Sporophyll extract of the present invention with a carrier and a binder such as polyvinylpyrrolidone, hydroxypropyl cellulose, or the like and then subjecting the mixture to wet granulation using a solvent such as water, ethanol, isopropanol, or the like or dry granulation using compressive force. Also, the tablet may be prepared by mixing the granule with a lubricant such as magnesium stearate or the like and then pressing the mixture using a tablet press.

In an embodiment, the pharmaceutical composition may be administered in the form of an oral preparation, an injection (e.g., intramuscular injection, intraperitoneal injection, intravenous injection, infusion, subcutaneous injection, implant), an inhalant, a nasal preparation, a vaginal preparation, a rectal preparation, a sublingual preparation, a transdermal preparation, a topical preparation, or the like according to the condition of a disease or subject to be treated, but the present invention is not limited thereto. Also, the pharmaceutical composition of the present invention may be prepared as a suitable dosage unit containing a pharmaceutically acceptable carrier, additive, or vehicle, which is commonly used and non-toxic, according to an administration route.

In an embodiment, the dose of the pharmaceutical composition may vary depending on the condition and weight of a patient, the severity of a disease, the form of a drug, an administration route, and an administration time, but may be properly selected by those skilled in the art. The dosage form, administration route, and administration mode of the pharmaceutical composition may be independent from each other and are not particularly limited, and any administration route and administration mode may be used as long as the pharmaceutical composition can reach the desired site. For example, the pharmaceutical composition according to the present invention may be used in the form of its pharmaceutically acceptable salt and may be used alone or in combination with other pharmaceutically active compounds as well as in appropriate combinations. Also, for example, the administration mode of the pharmaceutical composition according to the present invention may include not only intravenous administration, intraperitoneal administration, intramuscular administration, percutaneous administration, and subcutaneous administration but also application of the composition onto the diseased site, spraying, and inhalation, but the present invention is not limited thereto.

### EXPERIMENTAL EXAMPLES

Hereinafter, exemplary examples will be described to describe the present invention in further detail, but the present invention is not limited thereto.

### Experimental Example 1: Confirmation of Synergistic Effect According to Use of Oil Mixture

### (1) Preparation of Examples 1 to 3

Compositions according to the examples of the present invention were prepared using components described in the following Table 1. The content units of components shown in Table 1 are wt %.

TABLE 1

| Component | Example 1 | Example 2 | Example 3 |
|---|---|---|---|
| Lemongrass oil | 60 | 50 | 66.667 |
| Lavender oil | 30 | 37.5 | 25 |
| Ylang ylang oil | 10 | 12.5 | 8.333 |
| Lemongrass | 6:3:1 | 4:3:1 | 8:3:1 |
| oil:lavender | | | |
| oil:ylang ylang oil | | | |
| Total | 100 | 100 | 100 |

A specific preparation method is as follows.

Oil components were mixed and then uniformly stirred for 10 minutes to prepare antibacterial compositions of Examples 1 to 3.

### (2) Preparation of Comparative Examples 1 to 6

Compositions according to the comparative examples of the present invention were prepared using components described in the following Table 2. The content units of components shown in Table 2 are wt %.

TABLE 2

| | Comparative | Comparative | Comparative | Comparative | Comparative | Comparative |
|---|---|---|---|---|---|---|
| Component | Example 1 | Example 2 | Example 3 | Example 4 | Example 5 | Example 6 |
| Lemongrass oil | 100 | — | — | — | 85.715 | 66.667 |
| Lavender oil | — | 100 | — | 75 | — | 33.333 |
| Ylang ylang oil | — | — | 100 | 25 | 14.285 | — |
| Total | 100 | 100 | 100 | 100 | 100 | 100 |

A specific preparation method is the same as in Examples 1 to 3.

### (3) Minimum Inhibitory Concentration (MIC) Measurement

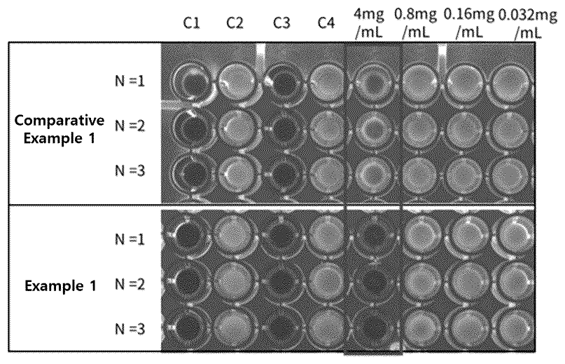

The antibacterial activities of Example 1 and Comparative Example 1 were evaluated through minimum inhibitory concentration (MIC), and results thereof are shown in Table 3 and FIG. 1.

An experimental method is as follows. 100 μl of 200% TSB was added to a 96-well plate, and then samples were prepared so that the concentration of oil to be treated was doubled, and 100 μl of each was dispensed into the 96-well plate. Afterward, each sample of Example 1 and Comparative Example 1 was adjusted so as to be 4000 ppm, 800 ppm, 160 ppm, and 32 ppm and cultured at 32.5° C. for 24 hours. After a predetermined period of time, the presence or absence of bacterial growth was determined based on the change in color (yellow indicates bacterial growth; colorlessness indicates bacterial growth inhibition). Control 1 (C1) is a medium in which the strain was not inoculated, Control 2 (C2) is a medium in which the antibacterial composition was not added after strain inoculation, Positive Control (C3) is a medium in which phenoxyethanol was added instead of the antibacterial composition, and Negative Control (C4) is a medium in which DMSO was added.

TABLE 3

| Classification | Minimum inhibitory concentration | |
|---|---|---|
| Example 1 | 800 to 4000 | ppm |
| Comparative Example 1 | more than 4000 | ppm |

As a result of the experiment, Example 1 using a mixture of three types of oils exhibited a yellow color at 800 ppm and colorlessness at 4000 ppm. Therefore, it was found that the minimum inhibitory concentration of Example 1 against Staphylococcus aureus was 800 to 4000 ppm.

Meanwhile, Comparative Example 1 using only lemongrass oil exhibited a yellow color even at 4000 ppm. Therefore, it was found that the minimum inhibitory concentration of Comparative Example 1 against Staphylococcus aureus exceeded 4000 ppm.

In other words, it was confirmed that Example 1 had improved antibacterial activity against Staphylococcus aureus compared to Comparative Example 1.

### (4) Paper Disk Diffusion Test

The antibacterial activities of Examples 1 to 3 and Comparative Examples 2 to 6 were evaluated through a paper disk diffusion test, and results thereof are shown in Table 4.

An experimental method is as follows. A Staphylococcus aureus strain was inoculated in tryptic soy agar using a 100% loop and cultured for 24 hours. Afterward, the cultured strain was suspended in 0.85% NaCl to a density of 1.0×10⁷ to prepare a bacterial solution. 100 μl of the prepared bacterial solution was dispensed into a TSA medium and then smeared with glass beads so that the number of bacteria was 1.0×10⁶. Each sample of the examples and comparative examples was dispensed into a paper disk at 40 μl/disk, and the sample was allowed to be sufficiently absorbed into the paper disk, then the resultant was placed on the medium in which the strain was smeared, and the plate treated with the sample was turned over and cultured at 32.5° C. for 24 hours. After the culturing, the diameters of the short and long sides of the growth inhibition zone around the disk were measured, and the average value was derived to evaluate antibacterial activity.

TABLE 4

| | Diameter of growth |
|---|---|
| Classification | inhibition zone (mm) |
| Example 1 | 43.7 |
| Example 2 | 43.07 |
| Example 3 | 44.3 |
| Comparative Example 2 | 25.8 |
| Comparative Example 3 | 24.8 |
| Comparative Example 4 | 24.7 |
| Comparative Example 5 | 30.1 |
| Comparative Example 6 | 37.4 |

As a result of the experiment, in the case of all of Examples 1 to 3 using a mixture of lemongrass oil, lavender oil, and ylang ylang oil, a growth inhibition zone diameter was measured to be 43 mm or more, and thus it was found that antibacterial activity was excellent.

Meanwhile, in the case of Comparative Examples 2 and 3 using only one of lemongrass oil, lavender oil, and ylang ylang oil, a growth inhibition zone diameter was measured to be less than 26 mm, and in the case of all of Comparative Examples 4 to 6 using only two types of oils, a growth inhibition zone diameter was measured to be less than 38 mm, and thus it was found that antibacterial activity was degraded compared to Examples 1 to 3.

### Experimental Example 2: Confirmation of Effect According to Weight Ratio of Oils

### (1) Preparation of Comparative Examples 7 to 13

Compositions according to the comparative examples of the present invention were prepared using components described in the following Table 5. The content units of components shown in Table 5 are wt %.

TABLE 5

| | Comparative | Comparative | Comparative | Comparative | Comparative | Comparative | Comparative |
|---|---|---|---|---|---|---|---|
| Component | Example 7 | Example 8 | Example 9 | Example 10 | Example 11 | Example 12 | Example 13 |
| Lemongrass oil | 75 | 50 | 50 | 69.232 | 42.857 | 56.25 | 56.25 |
| Lavender oil | 12.5 | 41.667 | 25 | 23.076 | 42.857 | 37.5 | 18.75 |
| Ylang ylang oil | 12.5 | 8.333 | 25 | 7.692 | 14.286 | 6.25 | 25 |
| Lemongrass | 6:1:1 | 6:5:1 | 2:1:1 | 9:3:1 | 3:3:1 | 9:6:1 | 9:3:4 |
| oil:lavender | | | | | | | |
| oil:ylang | | | | | | | |
| ylang oil | | | | | | | |
| Total | 100 | 100 | 100 | 100 | 100 | 100 | 100 |

A specific preparation method is the same as in Examples 1 to 3.

### (2) Paper Disk Diffusion Test

The antibacterial activities of Examples 1 to 3 and Comparative Examples 7 to 13 were evaluated in the same manner as in Experimental Example 1 through the paper disk diffusion test, and results thereof are shown in Table 6.

TABLE 6

| | Diameter of growth |
|---|---|
| Classification | inhibition zone (mm) |
| Example 1 | 43.7 |
| Example 2 | 43.07 |
| Example 3 | 44.3 |
| Comparative Example 7 | 29.0 |
| Comparative Example 8 | 27.0 |
| Comparative Example 9 | 31.4 |
| Comparative Example 10 | 29.2 |
| Comparative Example 11 | 34.5 |
| Comparative Example 12 | 36.8 |
| Comparative Example 13 | 28.2 |

As a result of the experiment, in the case of all of Examples 1 to 3 using lemongrass oil, lavender oil, and ylang ylang oil within the weight ratio range of the present invention, a growth inhibition zone diameter was measured to be 43 mm or more, and thus it was found that antibacterial activity was excellent.

In addition, in the case of all of Comparative Examples 7 to 13 using lemongrass oil, lavender oil, and ylang ylang oil outside the weight ratio range of the present invention, a growth inhibition zone diameter was measured to be less than 37 mm, and thus it was found that antibacterial activity was degraded compared to Examples 1 to 3.

The antibacterial composition according to the present invention can provide excellent antibacterial or antifungal effects against harmful bacteria that cause a symptom such as hair loss, acne, or dandruff by including lemongrass oil, lavender oil, and ylang ylang oil.

In addition, the antibacterial composition according to the present invention can alleviate a symptom such as hair loss, acne, or dandruff and promote hair growth.

Although a specific part of the content of the present invention has been described in detail, it will be obvious to those skilled in the art that this specific description is only a preferred embodiment and the scope of the present invention is not limited thereby. Therefore, the actual scope of the present invention will be defined by the appended claims and their equivalents.

1. An antibacterial composition comprising lemongrass oil, lavender oil, and ylang ylang oil.

2. The antibacterial composition of claim 1, wherein a weight ratio of the lavender oil and the lemongrass oil is 1:1.3 to 2.7.

3. The antibacterial composition of claim 1, wherein a weight ratio of the ylang ylang oil and the lemongrass oil is 1:4 to 8.

4. The antibacterial composition of claim 1, wherein a weight ratio of the ylang ylang oil and the lavender oil is 1:1.5 to 4.5.

5. The antibacterial composition of claim 1, wherein a weight ratio of the ylang ylang oil, the lavender oil, and the lemongrass oil is 1:1.5 to 4.5:4 to 8.

6. The antibacterial composition of claim 1, wherein the antibacterial composition has antibacterial activity against at least one of strains of genus Staphylococcus, genus Streptococcus, genus Malassezia, genus Propionibacterium, and genus Candida.

7. A cosmetic composition comprising the antibacterial composition of claim 1.

8. A composition for external use on the skin, comprising the antibacterial composition of claim 1.

9. A pharmaceutical composition for preventing or treating hair loss, comprising the antibacterial composition of claim 1.
